(19) United States
(12) Patent Application Publication
(10) Pub. No.: US 2021/0186850 A1
(43) Pub. Date: Jun. 24, 2021

(54) COMPOSITION COMPRISING ANIONIC, NON-IONIC AND AMPHOTERIC SURFACTANTS, AND CATIONIC ASSOCIATIVE POLYMERS

(71) Applicant: L'OREAL, Paris (FR)

(72) Inventors: Virginie LE CHAUX, SAINT-OUEN (FR); Emmanuelle LEBON-HIPOLITE, SAINT-OUEN (FR); Saber MALOUG, SAINT-OUEN (FR)

(21) Appl. No.: 17/252,465

(22) PCT Filed: Jun. 19, 2019
(86) PCT No.: PCT/EP2019/066248

§ 371 Date: Dec. 15, 2020

(30) Foreign Application Priority Data
Jun. 28, 2018 (FR) 1855839

(51) Int. Cl. A61K 8/87; A61Q 5/02; A61K 8/39; A61K 8/46; A61K 8/44; A61K 8/60; A61K 8/73; A61K 8/81; A61K 8/86

(52) U.S. Cl. CPC A61K 8/87; A61Q 5/02; A61K 8/39; A61K 8/463; A61K 2800/596; A61K 8/602; A61K 8/731; A61K 8/817; A61K 8/86; A61K 8/442

(57) ABSTRACT

The present invention relates to a composition intended for the cosmetic treatment of keratin fibres, in particular the hair, comprising anionic surfactants of polyoxy-alkylenated alkyl(amido)ether carboxylic acid type, additional anionic surfactants, non-ionic and amphoteric surfactants and cationic associative polymers. The invention also relates to a process for the cosmetic treatment of keratin fibres, in particular for washing and/or conditioning the hair, using such a composition, and also to a use employing said composition.

The present invention relates to a cosmetic composition, in particular a hair composition, and most particularly for cleansing and/or conditioning the hair, comprising a particular combination of anionic, non-ionic and amphoteric surfactants, and also one or more cationic associative polymers. The invention also relates to a process for the cosmetic treatment of keratin materials using said composition, and also to the use of said composition for the cosmetic treatment of keratin materials, in particular for conferring conditioning properties on said keratin materials.

Hair can generally be damaged and embrittled by the action of external atmospheric agents such as light and bad weather, and/or by mechanical or chemical treatments, such as brushing, combing, dyeing, bleaching, permanent-waving and/or relaxing. To remedy these drawbacks, it is now common practice to have recourse to hair compositions which make it possible to condition the hair after these treatments to give it in particular sheen, softness, suppleness, lightness, a natural feel and disentangling properties. These hair compositions may for example be care compositions, to be applied before or after a shampooing operation, or else compositions that are both washing and conditioning compositions, such as conditioning shampoos, for example. The latter generally have good washing power, but the cosmetic properties provided can still be improved; conditioning agents, most commonly silicone-based conditioning agents, are then generally included in these shampoos.

However, silicones are not always well liked by consumers, who are increasingly looking for compositions which make it possible to cleanse the hair while at the same time conditioning it, but which do not comprise silicones.

It has been proposed to replace silicones with various carbon-based conditioning agents, such as fatty alcohols or fatty esters; however, these hair compositions are not necessarily entirely satisfactory and can still be improved, in particular with regard to the depositing of the conditioning agents, in particular non-silicone conditioning agents, on keratin fibres.

There is thus a real need to develop cosmetic compositions, in particular hair compositions, intended for cleansing and conditioning keratin materials, in particular hair, which are capable of conferring thereon improved cosmetic properties, in particular in terms of disentangling, smooth feel and visual smoothness, suppleness and coating of the fibre, while at the same time retaining good washing power, in particular good foaming power (abundant foam, rapidly generated, of good quality).

This aim is achieved by the present invention, a subject of which is a cosmetic composition, preferably hair composition, comprising:

(i) one or more anionic surfactants chosen from polyoxyalkylenated alkyl(amido)ether carboxylic acids and salts thereof;
(ii) one or more anionic surfactants other than the polyoxyalkylenated alkyl(amido)ether carboxylic acid anionic surfactant(s) (i);
(iii) one or more non-ionic surfactants;
(iv) one or more amphoteric surfactants; and
(v) one or more cationic associative polymers,
with a (iv)/(ii) weight ratio greater than or equal to 0.5.

It was found that the composition according to the invention has satisfactory foaming power. It allows the production of an abundant, rapidly-generated foam, which spreads easily on keratin fibres and is easy to remove on rinsing.

Certain users of shampoos, in particular conditioning shampoos, may have more or less sensitized hair, i.e. hair that is generally damaged or embrittled by the action of external atmospheric agents such as light and bad weather, and/or the action of mechanical or chemical treatments such as brushing, combing, dyeing, bleaching, permanent-waving and/or relaxing. Thus, the composition according to the invention also makes it possible to improve the cosmetic properties imparted to the keratin fibres, especially to the hair, preferably sensitized hair. In particular, the composition according to the invention makes it possible to improve the disentangling and the suppleness of the hair, and also the smooth feel and visual smoothness of the hair, and the coating of the fibres. These properties make it possible to confer a good level of perception of care given to the hair.

The composition according to the invention also has the advantage of being stable on storage both at ambient temperature (20-25° C.) and at 45° C., especially as regards its visual appearance and/or its viscosity.

For the purposes of the present invention, the term “stable” is intended to mean a composition which, after two months of storage, shows no change in appearance, colour, odour or viscosity.

Preferably, the composition according to the invention is non-colouring.

According to the present invention, the term “non-colouring composition” means a composition not containing any dye for keratin fibres, such as direct dyes or oxidation dye precursors (bases and/or couplers). If they are present, their content does not exceed 0.005% by weight, relative to the total weight of the composition. Specifically, at such a content, only the composition would be dyed, i.e. no dyeing effect would be observed on the keratin fibres.

In the following text, and unless otherwise indicated, the limits of a range of values are included in that range, especially in the expressions “between” and “ranging from . . . to . . . ”.

Moreover, the expression “at least one” used in the present description is equivalent to the expression “one or more”, and may be replaced therewith.

### Anionic Surfactants

As indicated above, the composition according to the invention comprises at least one anionic surfactant of polyoxyalkylenated alkyl(amido)ether carboxylic acid type (i) and at least one anionic surfactant (ii) other than the anionic surfactant(s) (i).

Thus, for the purposes of the invention, the cosmetic composition comprises at least two different anionic surfactants.

The term “anionic surfactant” means a surfactant including, as ionic or ionizable groups, only anionic groups.

In the present description, a species is termed “anionic” when it bears at least one permanent negative charge or when it can be ionized into a negatively charged species, under the conditions of use of the composition of the invention (for example the medium or the pH) and not comprising any cationic charge.

### Polyoxyalkylenated Alkyl(Amido)Ether Carboxylic Acids (i)

The composition of the invention contains at least one anionic surfactant chosen from polyoxyalkylenated alkyl(amido)ether carboxylic acids and salts thereof, in particular those comprising from 2 to 50 alkylene oxide and in particular ethylene oxide groups.

The polyoxyalkylenated alkyl(amido)ether carboxylic acids that may be used are preferably chosen from those of formula (1):

R₁OC₂H₄OCH₂COOA (1)

in which:
R1 represents a linear or branched C₆-C₂₄ alkyl or alkenyl radical, a (C₈-C₉)alkylphenyl radical, a radical R2CONH—CH₂—CH₂— with R2 denoting a linear or branched C₉-C₂₁ alkyl or alkenyl radical; preferably, R1 is a C₈-C₂₀, preferably C₈-C₁₈, alkyl radical,
n is an integer or decimal number (average value) ranging from 2 to 24 and preferably from 2 to 10,
A denotes H, ammonium, Na, K, Li, Mg, Ca or a monoethanolamine or triethanolamine residue.

It is also possible to use mixtures of compounds of formula (1), in particular mixtures of compounds containing different groups R1.

The polyoxyalkylenated alkyl(amido)ether carboxylic acids that are particularly preferred are those of formula (1) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of compounds of formula (1) in which R denotes a C₁₂ alkyl radical, A denotes a hydrogen or sodium atom and n ranges from 2 to 10.

Use is preferably made of polyoxyalkylenated (C₆-C₂₄)alkyl ether carboxylic acids and salts thereof, polyoxyalkylenated (C₆-C₂₄)alkylamido ether carboxylic acids, in particular those comprising from 2 to 15 alkylene oxide groups, salts thereof, and mixtures thereof.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Among the commercial products that may preferably be used are the products sold by the company KAO under the names:

Akypo® NP 70 (R₁=nonylphenyl, n=7, A=H)
Akypo® NP 40 (R₁=nonylphenyl, n=4, A=H)
Akypo® OP 40 (R₁=octylphenyl, n=4, A=H)
Akypo® OP 80 (R₁=octylphenyl, n=8, A=H)
Akypo® OP 190 (R₁=octylphenyl, n=19, A=H)
Akypo® RLM 38 (R₁═(C₁₂-C₁₄)alkyl, n=4, A=H)
Akypo® RLM 38 NV (R₁═(C₁₂-C₁₄)alkyl, n=4, A=Na)
Akypo® RLM 45 CA (R₁═(C₁₂-C₁₄)alkyl, n=4.5, A=H)
Akypo® RLM 45 NV (R₁═(C₁₂-C₁₄)alkyl, n=4.5, A=Na)
Akypo® RLM 100 (R₁═(C₁₂-C₁₄)alkyl, n=10, A=H)
Akypo® RLM 100 NV (R₁═(C₁₂-C₁₄)alkyl, n=10, A=Na)
Akypo® RLM 130 (R₁═(C₁₂-C₁₄)alkyl, n=13, A=H)
Akypo® RLM 160 NV (R₁═(C₁₂-C₁₄)alkyl, n=16, A=Na),
or by the company Sandoz under the names:
Sandopan DTC-Acid (R₁═(C₁₃)alkyl, n=6, A=H)
Sandopan DTC (R₁═(C₁₃)alkyl, n=6, A=Na)
Sandopan LS 24 (R₁═(C₁₂-C₁₄)alkyl, n=12, A=Na)
Sandopan JA 36 (R₁═(C₁₃)alkyl, n=18, A=H),
and more particularly the products sold under the following names:
Akypo® RLM 45 (INCI: Laureth-5 carboxylic acid)

### Akypo® RLM 100

### Akypo® RLM 38.

Preferably, polyoxyalkylenated (C₆-C₂₄)alkyl ether carboxylic acids and salts thereof are used.

The composition according to the invention preferably comprises said polyoxyalkylenated alkyl(amido)ether carboxylic acid(s) and/or salts thereof in a total amount ranging from 0.1% to 30% by weight, preferably from 0.5% to 25% by weight, better still from 1% to 20% by weight and preferentially from 1.5% to 10% by weight, relative to the total weight of the composition.

### Additional Anionic Surfactants (ii)

As indicated above, the composition according to the invention comprises at least one additional anionic surfactant other than the polyoxyalkylenated alkyl(amido)ether carboxylic acids and salts thereof (i) described above.

Preferably, the additional anionic surfactants (ii) used in the composition according to the invention are chosen from anionic surfactants comprising in their structure one or more sulfate and/or sulfonate and/or phosphate and/or carboxylate groups, and/or mixtures thereof, preferably sulfate groups.

The anionic surfactant(s) (ii) may be oxyethylenated and/or oxypropylenated. The total average number of ethylene oxide (EO) and/or propylene oxide (PO) groups may then range from 1 to 50 and especially from 1 to 10.

The carboxylic anionic surfactants that may be used thus comprise at least one carboxylic or carboxylate function.

They can be chosen from the following compounds: acylglycinates, acyllactylates, acylsarcosinates, acylglutamates; alkyl-D-galactoside-uronic acids; and also the salts of these compounds; the alkyl and/or acyl groups of these compounds comprising from 6 to 30 carbon atoms, in particular from 12 to 28, better still from 14 to 24 or even from 16 to 22 carbon atoms; these compounds possibly being polyoxyalkylenated, in particular polyoxyethylenated, and then preferably comprising from 1 to 50 ethylene oxide units, better still from 1 to 10 ethylene oxide units.

Use may also be made of the C₆-C₂₄ alkyl monoesters of polyglycoside-polycarboxylic acids, such as C₆-C₂₄ alkyl polyglycoside-citrates, C₆-C₂₄ alkyl polyglycosidetartrates and C₆-C₂₄ alkyl polyglycoside-sulfosuccinates, and salts thereof.

Preferentially, the carboxylic anionic surfactants are chosen, alone or as a mixture, from:
acylglutamates, especially of C₆-C₂₄ or even C₁₂-C₂₀, such as stearoylglutamates, and in particular disodium stearoylglutamate;
acylsarcosinates, in particular of C₆-C₂₄ or even C₁₂-C₂₀, such as palmitoylsarcosinates, and in particular sodium palmitoylsarcosinate;
acyllactylates, in particular of C₁₂-C₂₈ or even C₁₄-C₂₄, such as behenoyllactylates, and in particular sodium behenoyllactylate;
C₆-C₂₄ and especially C₁₂-C₂₀ acylglycinates;
   in particular in the form of alkali metal or alkaline-earth metal, ammonium or amino alcohol salts.

The sulfonate anionic surfactants that may be used comprise at least one sulfonate function.

They may be chosen from the following compounds: alkylsulfonates, alkylamidesulfonates, alkylarylsulfonates, alpha-olefin sulfonates, paraffin sulfonates, alkylsulfosuccinates, alkyl ether sulfosuccinates, alkylamide sulfosuccinates, alkylsulfoacetates, N-acyltaurates, acylisethionates; alkylsulfolaurates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units, better still from 2 to 10 ethylene oxide units.

Preferentially, the sulfonate anionic surfactants are chosen, alone or as a mixture, from:
C₆-C₂₄ and especially C₁₂-C₂₀ olefin sulfonates;
C₆-C₂₄ and especially C₁₂-C₂₀ alkylsulfosuccinates, especially laurylsulfosuccinates;
C₆-C₂₄ and especially C₁₂-C₂₀ alkyl ether sulfosuccinates;
(C₆-C₂₄) acylisethionates and preferably (C₁₂-C₁₈) acylisethionates;
   in particular in the form of alkali metal or alkaline-earth metal, ammonium or amino alcohol salts.

The sulfate anionic surfactants that may be used comprise at least one sulfate function.

They may be chosen from the following compounds: alkyl sulfates, alkyl ether sulfates, alkylamido ether sulfates, alkylarylpolyether sulfates, monoglyceride sulfates; and the salts of these compounds; the alkyl groups of these compounds including from 6 to 30 carbon atoms, especially from 12 to 28, better still from 14 to 24 or even from 16 to 22 carbon atoms; the aryl group preferably denoting a phenyl or benzyl group; these compounds may be polyoxyalkylenated, especially polyoxyethylenated, and then preferably comprising from 1 to 50 ethylene oxide units, better still from 2 to 10 ethylene oxide units.

Preferentially, the sulfate anionic surfactants are chosen, alone or as a mixture, from:
alkyl sulfates, in particular of C₆-C₂₄ or even C₁₂-C₂₀,
alkyl ether sulfates, in particular of C₆-C₂₄ or even C₁₂-C₂₀, preferably comprising from 2 to 20 ethylene oxide units;
   in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium or calcium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the additional anionic surfactants (ii) are chosen, alone or as a mixture, from:
C₆-C₂₄ and especially C₁₂-C₂₀ alkyl sulfates;
C₆-C₂₄ and especially C₁₂-C₂₀ alkyl ether sulfates; preferably comprising from 2 to 20 ethylene oxide units;
C₆-C₂₄ and especially C₁₂-C₂₀ olefin sulfonates;
C₆-C₂₄ and especially C₁₂-C₂₀ alkylsulfosuccinates, especially laurylsulfosuccinates;
C₆-C₂₄ and especially C₁₂-C₂₀ alkyl ether sulfosuccinates;
(C₆-C₂₄) acylisethionates and preferably (C₁₂-C₁₈)acylisethionates;
C₆-C₂₄ and especially C₁₂-C₂₀ acylsarcosinates; in particular palmitoylsarcosinates;
C₆-C₂₄ and especially C₁₂-C₂₀ acylglutamates;
C₆-C₂₄ and especially C₁₂-C₂₀ acylglycinates;
   in particular in the form of alkali metal or alkaline-earth metal, ammonium or amino alcohol salts.

Among the anionic surfactants (ii), use is preferably made of one or more sulfate-based anionic surfactants, and/or one or more sulfonate-based anionic surfactants. Preferably, the anionic surfactant(s) (ii) are in the form of salts, and in particular alkaline salts, especially sodium salts, ammonium salts, amine salts, including amino alcohol salts, and/or magnesium salts. These salts preferably comprise from 2 to 5 ethylene oxide groups.

Among these salts, sodium, triethanolamine, magnesium or ammonium (C₁₂-C₁₄)alkyl sulfates and/or sodium, ammonium or magnesium (C₁₂-C₁₄)alkyl ether sulfates, which are oxyethylenated, for example with 1 to 10 mol of ethylene oxide, are more preferably used.

Among the sulfonate-based surfactants, use is preferably made of C₁₂-C₂₀ olefin sulfonates, such as sodium C₁₄-C₁₆ olefin sulfonates.

Better still, the anionic surfactant(s) (ii) are chosen from sodium, ammonium or magnesium (C₁₂-C₁₄)alkyl ether sulfates oxyethylenated with 2.2 mol of ethylene oxide, as sold under the name Texapon N702 by the company Cognis.

The anionic surfactant(s) (ii) may be present in the composition according to the invention in a total content ranging from 3% to 20% by weight, preferably in a content ranging from 3.5% to 18% by weight and better still from 4% to 15% by weight, even better still from 5% to 10% by weight, relative to the total weight of the composition.

According to a particular embodiment, the total content of anionic surfactants in the composition according to the invention is between 3% and 35% by weight, preferably between 5% and 25% by weight, and preferentially between 7% and 20% by weight, relative to the total weight of the composition.

Non-Ionic Surfactants (iii)

As indicated above, the composition comprises one or more non-ionic surfactants.

The non-ionic surfactants may be chosen from:
alcohols, α-diols and (C1-20)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or else these compounds comprising at least one fatty chain comprising from 8 to 40 carbon atoms and in particular from 16 to 30 carbon atoms; in particular, oxyethylenated alcohols comprising at least one saturated or unsaturated, linear or branched C8 to C40 alkyl chain, comprising from 1 to 100 mol of ethylene oxide, preferably from 2 to 50 and more particularly from 2 to 40 mol of ethylene oxide and comprising one or two fatty chains;
condensates of ethylene oxide and propylene oxide with fatty alcohols;
polyethoxylated fatty amides preferably containing from 2 to 30 ethylene oxide units, polyglycerolated fatty amides including on average from 1 to 5 and in particular from 1.5 to 4 glycerol groups;
ethoxylated fatty acid esters of sorbitan, preferably containing from 2 to 40 mol of ethylene oxide units;
fatty acid esters of sucrose;
polyoxyalkylenated, preferably polyoxyethylenated, fatty acid esters containing from 2 to 150 mol of ethylene oxide, including oxyethylenated plant oils;
N—(C6-24 alkyl)glucamine derivatives;
amine oxides such as (C10-14 alkyl)amine oxides or N—(C10-14 acyl)aminopropylmorpholine oxides;

In one preferred embodiment, the composition according to the invention comprises one or more non-ionic surfactants of alkyl(poly)glycoside type, in particular represented by the following general formula: R1O—(R2O)t-(G)v in which:
R1 represents a linear or branched alkyl or alkenyl radical including 6 to 24 carbon atoms and especially 8 to 18 carbon atoms, or an alkylphenyl radical of which the linear or branched alkyl radical includes 6 to 24 carbon atoms and especially 8 to 18 carbon atoms,
R2 represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.

Preferably, the alkyl(poly)glycoside surfactants are compounds of the formula described above in which:
R1 denotes a linear or branched, saturated or unsaturated alkyl radical including from 8 to 18 carbon atoms,
R2 represents an alkylene radical including 2 to 4 carbon atoms,
t denotes a value ranging from 0 to 3 and preferably equal to 0,
G denotes glucose, fructose or galactose, preferably glucose,
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C₈/C₁₆ alkyl(poly)glucosides 1,4, and in particular decyl glucosides and caprylyl/capryl glucosides, are most particularly preferred. Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of C₈/C₁₆ alkyl(poly)glycosides 1,4, in particular as an aqueous 53% solution, such as those sold by Cognis under the reference Plantacare® 818 UP.

Preferentially, the non-ionic surfactants are chosen from, alone or as a mixture, (C₆-C₂₄ alkyl)(poly)glycosides, and more particularly (C₈-C₁₈ alkyl)(poly)glycosides.

Preferably, the non-ionic surfactant(s) are present in the composition according to the invention in a total content ranging from 0.1% to 15% by weight, preferentially ranging from 0.2% to 10% by weight, in particular ranging from 0.5% to 8% by weight, relative to the total weight of the composition.

Preferably, the non-ionic surfactant(s) of alkyl(poly)glycoside type are present in the composition according to the invention in a total content ranging from 0.1% to 15% by weight, preferentially ranging from 0.2% to 10% by weight, in particular ranging from 0.5% to 8% by weight, relative to the total weight of the composition.

### Amphoteric Surfactants (iv)

The composition according to the invention also comprises one or more amphoteric surfactants.

In particular, the amphoteric or zwitterionic surfactant(s) are non-silicone surfactants.

They may especially be optionally quaternized secondary or tertiary aliphatic amine derivatives, in which the aliphatic group is a linear or branched chain containing from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

Among the optionally quaternized derivatives of secondary or tertiary aliphatic amines that may be used, as defined above, mention may also be made of the compounds having the respective structures (II) and (III) below:

Rₐ—CONHCH₂CH₂—N⁺(R_{b})(R_{c})—CH₂COO⁻,M⁺,X⁻ (II)

in which:
Rₐ represents a C₁₀ to C₃₀ alkyl or alkenyl group derived from an acid RₐCOOH preferably present in hydrolyzed coconut kernel oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

R_{a′}—CONHCH₂CH₂—N(B)(B′) (III)

in which:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid Rₐ—COOH which is preferably present in coconut kernel oil or in hydrolyzed linseed oil, or an alkyl group, especially a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C₂M Concentrate.

Use may also be made of compounds of formula (IV):

R_{a″}—NHCH(Y″)—(CH₂)ₙCONH(CH₂)_{n′}—N(R_{d})(Rₑ) (IV)

in which:
Y″ represents the group —COOH, —COOZ″ or —CH₂—CH(OH)SO₃H or the group CH₂CH(OH)SO₃—Z″;
R_{d} and Rₑ represent, independently of one another, a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid R_{a″}—COOH which is preferably present in coconut kernel oil or in hydrolyzed linseed oil;
n and n′ denote, independently of one another, an integer ranging from 1 to 3.

Mention may be made, among the compounds of formula (IV), of the compound classified in the CTFA dictionary under the name sodium diethylaminopropyl cocoaspartamide and sold by the company Chimex under the name Chimexane HB. These compounds may be used alone or as mixtures.

Among the amphoteric or zwitterionic surfactants, use is preferably made of (C₈-C₂₀)alkylbetaines such as cocoylbetaine, (C₈-C₂₀)alkylamido(C₃-C₈)alkylbetaines such as cocamidopropylbetaine, and mixtures thereof, and the compounds of formula (IV) such as the sodium salt of diethylaminopropyl laurylaminosuccinamate (INCI name: sodium diethylaminopropyl cocoaspartamide).

Preferentially, the amphoteric or zwitterionic surfactants are chosen from (C₈-C₂₀)alkylamido(C₃-C₈)alkylbetaines such as cocamidopropylbetaine.

Preferably, the amphoteric surfactant(s) are present in the composition according to the invention in a total content ranging from 3% to 20% by weight, preferentially in a content ranging from 3.5% to 15% by weight and better still from 4% to 10% by weight, relative to the total weight of the composition.

In the compositions according to the invention, the weight ratio of the total content of amphoteric surfactants (iv) to the total content of anionic surfactants (ii) different from the polyoxyalkylenated alkyl(amido)ether carboxylic acid anionic surfactants (i) is greater than or equal to 0.5, preferably greater than or equal to 0.6, in particular greater than or equal to 0.8. It may thus be between 0.5 and 3.0, in particular between 0.6 and 2.0, and better still between 0.7 and 1.5.

### Cationic Associative Polymers (v)

The composition according to the invention also comprises one or more cationic associative polymers.

These polymers are not silicone polymers, that is to say they do not comprise a silicon atom (Si).

The term “associative polymer” is intended to mean an amphiphilic polymer that is capable, in an aqueous medium, of reversibly combining with itself or with other molecules. It generally comprises, in its chemical structure, at least one hydrophilic region or group and at least one hydrophobic region or group. In particular, the hydrophobic group may be a fatty hydrocarbon-based chain such as a linear or branched alkyl, linear or branched arylalkyl or linear or branched alkylaryl group comprising at least 8 carbon atoms, preferably 8 to 30 carbon atoms, better still from 12 to 24 carbon atoms.

The term “cationic polymer” is intended to mean any polymer comprising cationic groups and/or groups that can be ionized into cationic groups and not comprising any anionic groups and/or groups that can be ionized into anionic groups.

Among the cationic associative polymers, mention may be made of, alone or as a mixture:
(A) cationic associative polyurethanes, which can be represented by general formula (Ia) below:

R—X—(P)n-[L-(Y)m]r-L′-(P′)p—X′—R′

in which:
R and R′, which may be identical or different, represent a hydrophobic group or a hydrogen atom;
X and X′, which may be identical or different, represent a group comprising an amine function optionally bearing a hydrophobic group, or alternatively the group L″;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100 inclusive, preferably between 1 and 50 inclusive and in particular between 1 and 25 inclusive;
n, m and p are each, independently of one another, between 0 and 1000 inclusive; the molecule containing at least one protonated or quaternized amine function and at least one hydrophobic group.

Preferably, the only hydrophobic groups are the groups R and R′ at the chain ends.

One preferred family of cationic associative polyurethanes is the one corresponding to formula (Ia) described above in which:

R and R′ both independently represent a hydrophobic group,
X and X′ each represent a group L″,
n and p are integers which are between 1 and 1000 inclusive, and L, L′, L″, P, P′, Y and m have the meaning indicated above.

Another preferred family of cationic associative polyurethanes is the one corresponding to formula (Ia) above in which:
n=p=0 (the polymers do not comprise units derived from a monomer containing an amine function, incorporated into the polymer during the polycondensation),
the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate.

Yet another preferred family of cationic associative polyurethanes is the one corresponding to formula (Ia) above in which:

R and R′ both independently represent a hydrophobic group,
X and X′ both independently represent a group comprising a quaternary amine,
n=p=0, and
L, L′, Y and m have the meaning indicated above.

The number-average molecular weight (Mn) of the cationic associative polyurethanes is preferably between 400 and 500 000 inclusive, in particular between 1000 and 400 000 inclusive and ideally between 1000 and 300 000 inclusive. The term “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When it denotes a hydrocarbon-based radical, the hydrophobic group comprises at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote a group comprising a tertiary or quaternary amine, X and/or X′ may represent one of the following formulae:

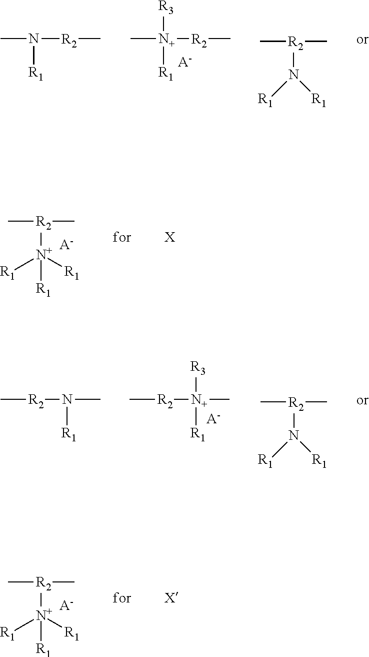

in which:
R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;
R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P;
A⁻ is a physiologically acceptable anionic counterion such as a halide, for instance a chloride or bromide, or a mesylate.

The groups L, L′ and L″ represent a group of formula:

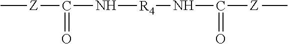

in which:
Z represents —O—, —S— or —NH—; and
R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

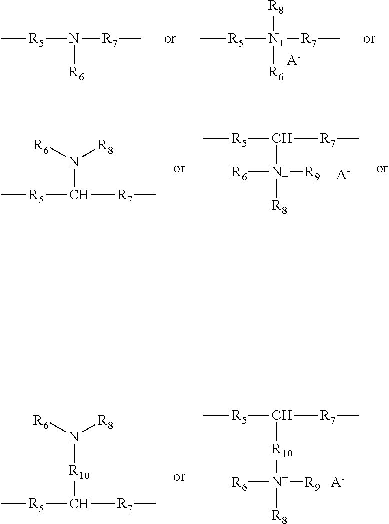

in which:
R₅ and R₇ have the same meanings as R₂ defined above;
R₆, R and R₉ have the same meanings as R₁ and R₃ defined above;
R₁₀ represents a linear or branched, optionally unsaturated alkylene group possibly containing one or more heteroatoms chosen from N, O, S and P;
and A⁻ is a physiologically acceptable anionic counterion such as a halide, for instance chloride or bromide, or mesylate.

As regards the meaning of Y, the term “hydrophilic group” is intended to mean a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. Preferentially, the hydrophilic compound is a polyether and in particular a poly(ethylene oxide) or poly(propylene oxide).

The cationic associative polyurethanes of formula (Ia) according to the invention are formed from diisocyanates and from various compounds bearing functions containing a labile hydrogen. The functions containing a labile hydrogen may be alcohol, primary or secondary amine or thiol functions, giving, after reaction with the diisocyanate functions, polyurethanes, polyureas and polythioureas, respectively. The term “polyurethanes” in the present invention encompasses these three types of polymer, namely polyurethanes per se, polyureas and polythioureas, and also copolymers thereof.

A first type of compound involved in the preparation of the polyurethane of formula (Ia) is a compound comprising at least one unit bearing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae: HZ—(P)ₙ—ZH, or HZ—(P′)ₚ—ZH, in which Z, P, P′, n and p are as defined above.

By way of example, mention may be made of N-methyldiethanolamine, N-tert-butyldiethanolamine and N-sulfoethyldiethanolamine.

The second compound involved in the preparation of the polyurethane of formula (Ia) is a diisocyanate corresponding to the formula:

O═C═N—R₄—N═C═O

in which R₄ is defined above.

By way of example, mention may be made of methylenediphenyl diisocyanate, methylenecyclohexane diisocyanate, isophorone diisocyanate, tolulene diisocyanate, naphthalene diisocyanate, butane diisocyanate and hexane diisocyanate.

A third compound involved in the preparation of the polyurethane of formula (Ia) is a hydrophobic compound intended to form the terminal hydrophobic groups of the polymer of formula (Ia).

This compound is formed from a hydrophobic group and a function containing a labile hydrogen, for example a hydroxyl, primary or secondary amine, or thiol function.

By way of example, this compound may be a fatty alcohol such as stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (Ia) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine, or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, by way of example, of polyethers, sulfonated polyesters and sulfonated polyamides, or a mixture of these polymers. Preferentially, the hydrophilic compound is a polyether and in particular a poly(ethylene oxide) or poly(propylene oxide).

The hydrophilic group termed Y in formula (Ia) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(B) quaternized cellulose derivatives, and in particular quaternized celluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferably, mention may be made of quaternized hydroxyethylcelluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferentially, mention may be made of the hydroxyethylcelluloses of formula (Ib):

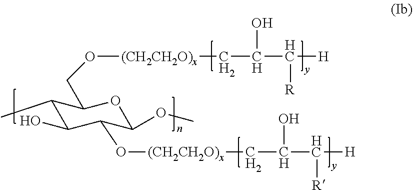

in which:
R represents an ammonium group RaRbRcN⁺-, Q⁻ in which Ra, Rb and Rc, which may be identical or different, represent a hydrogen atom or a linear or branched C₁-C₃₀ alkyl, and Q-represents an anionic counterion such as a halide, for instance a chloride or bromide; preferably an alkyl;
R′ represents an ammonium group R′aR′bR′cN⁺—, Q′⁻ in which R′a, R′b and R′c, which may be identical or different, represent a hydrogen atom or a linear or branched C₁-C₃₀ alkyl, and Q′⁻ represents an anionic counterion such as a halide, for instance a chloride or bromide; preferably an alkyl:
it being understood that at least one of the Ra, Rb, Rc, R′a, R′b and R′c radicals represents a linear or branched C₈-C₃₀ alkyl;
n, x and y, which may be identical or different, represent an integer between 1 and 10000.

Preferably, in formula (Ib), at least one of the Ra, Rb, Rc, R′a, R′b and R′c radicals represents a linear or branched C₈-C₃₀, better still C₁₀-C₂₄, or even C₁₀-C₁₄, alkyl; mention may in particular be made of the dodecyl radical (C₁₂). Preferably, the other radical(s) represent a linear or branched C₁-C₄ alkyl, in particular methyl.

Preferably, in formula (Ib), just one of the Ra, Rb, Rc, R′a, R′b and R′c radicals represents a linear or branched C₈-C₃₀, better still C₁₀-C₂₄, or even C₁₀-C₁₄, alkyl; mention may in particular be made of the dodecyl radical (C₁₂). Preferably, the other radicals represent a linear or branched C₁-C₄ alkyl, in particular methyl.

Even better still, R may be a group chosen from —N⁺(CH₃)₃, Q′⁻ and —N⁺(C₁₂H₂₅)(CH₃)₂, Q′⁻, preferably an —N⁺(CH₃)₃, Q′⁻ group.

Even better still, R′ may be an —N⁺(C₁₂H₂₅)(CH₃)₂, Q′⁻ group.

The aryl radicals preferably denote phenyl, benzyl, naphthyl or anthryl groups.

Mention may particularly be made of the following polymers having the INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (Ib) in which R represents trimethylammonium halide and R′ represents dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL100, SL-60, SL-30 and SL-5, from the company Amerchol/Dow Chemical.

More particularly, the polymers of formula (Ib) are those of which the viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs;
(C) cationic polyvinyllactams, in particular those comprising:
a) at least one monomer of vinyllactam or alkylvinyllactam type;
b) at least one monomer of structure (Ic) or (IIc) below:

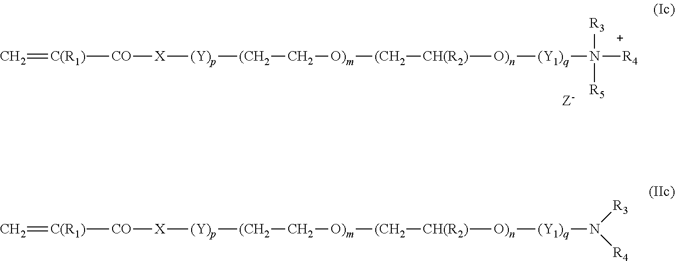

in which:
X denotes an oxygen atom or an NR radical,
R₁ and R₆ denote, independently of one another, a hydrogen atom or a linear or branched C₁-C₅ alkyl radical,
R₂ denotes a linear or branched C₁-C₄ alkyl radical,
R₃, R₄ and R₅ denote, independently of one another, a hydrogen atom, a linear or branched C₁-C₃₀ alkyl radical or a radical of formula (IIc):

—(Y₂)ᵣ—(CH₂—CH(R₇)—O)ₓ—R₈ (IIIc)
Y, Y₁ and Y₂ denote, independently of one another, a linear or branched C₂-C₁₆ alkylene radical,
R₇ denotes a hydrogen atom, or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of one another, 0 or 1,
m and n denote, independently of one another, an integer ranging from 0 to 100 inclusive,
x denotes an integer ranging from 1 to 100 inclusive,
Z denotes an anionic counterion of an organic or mineral acid, such as a halide,
   for instance chloride or bromide, or mesylate;
   with the proviso that:
at least one of the substituents R₃, R₄, R₅ or R₈ denotes a linear or branched C₉-C₃₀ alkyl radical,
if m and/or n is other than zero, then q is equal to 1,
if m=n=0, then p or q equals 0.

The cationic poly(vinyllactam) polymers according to the invention may be crosslinked or noncrosslinked and may also be block polymers.

Preferably, the counterion Z⁻ of the monomers of formula (Ic) is chosen from halide ions, phosphate ions, the methosulfate ion and the tosylate ion.

Preferably, R₃, R₄ and R₅ denote, independently of one another, a hydrogen atom or a linear or branched C₁-C₃₀ alkyl radical.

More preferentially, the monomer b) is a monomer of formula (Ic) for which, preferentially, m and n are equal to zero.

The vinyllactam or alkylvinyllactam monomer is preferably a compound of structure (IVc):

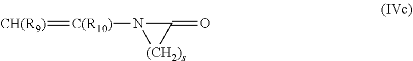

in which:
s denotes an integer ranging from 3 to 6,
R₉ denotes a hydrogen atom or a linear or branched C₁-C₅ alkyl radical,
R₁₀ denotes a hydrogen atom or a linear or branched C₁-C₅ alkyl radical, with the proviso that at least one of the radicals R₉ and R₁₀ denotes a hydrogen atom.

Even more preferentially, the monomer (IVc) is vinylpyrrolidone.

The cationic poly(vinyllactam) polymers according to the invention may also contain one or more additional monomers, preferably cationic or non-ionic monomers.

As compounds that are particularly preferred, mention may be made of the following terpolymers comprising at least:

a) a monomer of formula (IVc),
b) a monomer of formula (Ic) in which p=1, m=n=q=0, R₃ and R₄ denote, independently of one another, a hydrogen atom or a C₁-C₅ alkyl radical and R₅ denotes a linear or branched C₉-C₂₄ alkyl radical, and
c) a monomer of formula (IIc) in which p=1, m=n=q=0, and R₃ and R₄ denote, independently of one another, a hydrogen atom or a linear or branched C₁-C₅ alkyl radical.

Even more preferentially, terpolymers comprising, by weight, 40% to 95% of monomer (a), 0.1% to 55% of monomer (c) and 0.25% to 50% of monomer (b) will be used. Such polymers are described, in particular, in patent application WO00/68282.

As cationic poly(vinyllactam) polymers according to the invention, use is in particular made of:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylmethacrylamidopropylammonium chloride terpolymer is in particular provided by the company ISP under the names Styleze W10 or Styleze W20L® (INCI name Polyquaternium-55).

The weight-average molecular weight (Mw) of the cationic poly(vinyllactam) polymers is preferably between 500 and 20 000 000, more particularly between 200 000 and 2 000 000 and preferentially between 400 000 and 800 000;
(D) the cationic polymers obtained by polymerization of a monomer mixture comprising one or more vinyl monomers substituted with one or more amino groups, one or more hydrophobic non-ionic vinyl monomers, and one or more associative vinyl monomers, as described in patent application WO 2004/024779.

Among these polymers, mention may more particularly be made of the products of the polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide unit),
a 30/5 polyethylene glycol/polypropylene glycol allyl ether,
a hydroxy(C₂-C₆ alkyl) methacrylate, and
an ethylene glycol dimethacrylate.

Such a polymer is for example the compound sold by the company Lubrizol under the name Carbopol Aqua CC® and which corresponds to the INCI name Polyacrylate-1 crosspolymer.

Preferably, the composition comprises one or more associative cationic polymers chosen from the cationic polymers (B) derived from quaternized cellulose, particularly chosen from the hydroxyethylcelluloses of formula (Ib) and even better still polyquaternium-67.

The composition according to the invention may comprise the cationic associative polymer(s) in a total amount of between 0.01% and 8% by weight, in particular from 0.05% to 5% by weight and preferentially from 0.1% to 2% by weight, relative to the total weight of the composition.

Preferably, the composition according to the invention comprises one or more associative cationic polymers chosen from the cationic polymers (B) derived from quaternized cellulose, particularly chosen from the hydroxyethylcelluloses of formula (Ib) and even better still polyquaternium-67, in a total amount of preferably between 0.01% and 8% by weight, in particular from 0.05% to 5% by weight, preferentially from 0.1% to 2% by weight, relative to the total weight of the composition.

### Optional Cationic or Amphoteric Polymers

The cosmetic composition may also optionally comprise one or more cationic polymers, preferably with a cationic charge density of greater than or equal to 3 milliequivalents/gram (meq/g) and/or one or more amphoteric polymers. These polymers are different from the associative polymers described above (they are non-associative) and are non-silicone polymers (containing no silicon atoms Si).

The cationic charge density of a polymer corresponds to the number of moles of cationic charges per unit mass of polymer under conditions in which it is totally ionized. It may be determined by calculation if the structure of the polymer is known, i.e. the structure of the monomers constituting the polymer and their molar proportion or weight proportion. It may also be determined experimentally by the Kjeldahl method.

For the purposes of the present invention, the expression “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers that may be used preferably have a weight-average molar mass (Mw) of between 500 and 5×10⁶ approximately and preferably between 103 and 3×10⁶ approximately.

Among the cationic polymers, mention may be made more particularly of:

(1) homopolymers or copolymers derived from acrylic or methacrylic esters or amides and comprising at least one of the units of following formulae:

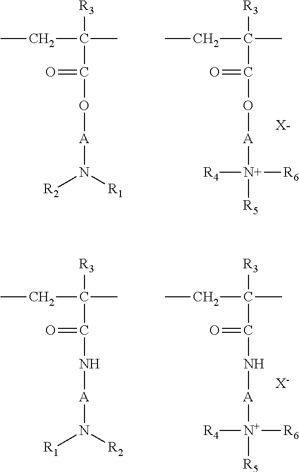

in which:
R3, which may be identical or different, denote a hydrogen atom or a CH3 radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R4, R5 and R6, which may be identical or different, represent an alkyl group containing from 1 to 18 carbon atoms or a benzyl radical, preferably an alkyl group containing from 1 to 6 carbon atoms;
R1 and R2, which may be identical or different, represent a hydrogen atom or an alkyl group containing from 1 to 6 carbon atoms, preferably methyl or ethyl;
X denotes an anion derived from a mineral or organic acid, such as a methosulfate anion or a halide such as chloride or bromide.

The copolymers of family (1) may also contain one or more units derived from comonomers that may be chosen from the family of acrylamides, methacrylamides, diacetone acrylamides, acrylamides and methacrylamides substituted on the nitrogen with lower (C1-C4) alkyls, acrylic or methacrylic acid esters, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as that sold under the name Hercofloc by the company Hercules,
copolymers of acrylamide and of methacryloyloxyethyltrimethylammonium chloride, such as those sold under the name Bina Quat P 100 by the company Ciba Geigy,
the copolymer of acrylamide and of methacryloyloxyethyltrimethylammonium methosulfate, such as that sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573;
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as those sold under the name Styleze CC 10 by ISP;
quaternized vinylpyrrolidone/dimethylaminopropylmethacrylamide copolymers such as the product sold under the name Gafquat HS 100 by the company ISP,
preferably crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.
   (2) cationic polysaccharides, in particular cationic celluloses and galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are in particular described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described in particular in patent U.S. Pat. No. 4,131,576, and mention may be made of hydroxyalkyl celluloses, for instance hydroxymethyl, hydroxyethyl or hydroxypropyl celluloses grafted, in particular, with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are in particular sold under the names Jaguar C13 S, Jaguar C 15, Jaguar C 17 and Jaguar C162 by the company Rhodia.

(3) polymers constituted of piperazinyl units and of divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized.
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.
(6) polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.
(7) cyclopolymers of alkyldiallylamine or of dialkyldiallylammonium, such as the homopolymers or copolymers containing, as main constituent of the chain, units corresponding to formula (I) or (II):

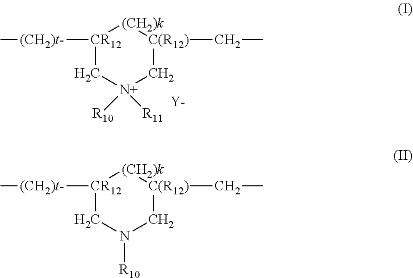

in which
k and t are equal to 0 or 1, the sum k+t being equal to 1;
R₁₂ denotes a hydrogen atom or a methyl radical;
R₁₀ and R₁₁, independently of each other, denote a C₁-C₆ alkyl group, a C₁-C₅ hydroxyalkyl group, a C₁-C₄ amidoalkyl group; or alternatively R₁₀ and R₁₁ may denote, together with the nitrogen atom to which they are attached, a heterocyclic group such as piperidyl or morpholinyl; R₁₀ and R₁₁, independently of each other, preferably denote a C₁-C₄ alkyl group;
Y⁻ is an anion such as bromide, chloride, acetate, borate, citrate, tartrate, bisulfate, bisulfite, sulfate or phosphate.

Mention may be made more particularly of the homopolymer of dimethyldiallylammonium salts (for example chloride) for example sold under the name Merquat 100 by the company Nalco and the copolymers of diallyldimethylammonium salts (for example chloride) and of acrylamide, sold in particular under the name Merquat 550 or Merquat 7SPR.

(8) quaternary diammonium polymers comprising repeating units of formula:

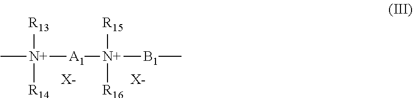

in which:
R₁₃, R₁₄, R₁₅ and R₁₆, which may be identical or different, represent aliphatic, alicyclic or arylaliphatic radicals comprising from 1 to 20 carbon atoms or C₁-C₁₂ hydroxyalkyl aliphatic radicals,
   or else R₁₃, R₁₄, R₁₅ and R₁₆, together or separately, form, with the nitrogen atoms to which they are attached, heterocycles optionally comprising a second non-nitrogen heteroatom;
   or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group, where R₁₇ is an alkylene and D is a quaternary ammonium group;
A₁ and B₁ represent linear or branched, saturated or unsaturated, divalent polymethylene groups comprising from 2 to 20 carbon atoms, which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
   it being understood that A₁, R₁₃ and R₁₅ can form, with the two nitrogen atoms to which they are attached, a piperazine ring;
   in addition, if A₁ denotes a linear or branched, saturated or unsaturated alkylene or hydroxyalkylene radical, B₁ may also denote a group (CH₂)n-CO-D-OC—(CH₂)p- with n and p, which may be identical or different, being integers ranging from 2 to 20, and D denoting:
a) a glycol residue of formula —O—Z—O—, in which Z denotes a linear or branched hydrocarbon-based radical or a group corresponding to one of the following formulae: —(CH₂CH₂O)x-CH₂CH₂— and —[CH₂CH(CH)O]y-CH₂CH(CH₃)—, in which x and y denote an integer from 1 to 4, representing a defined and unique degree of polymerization or any number from 1 to 4 representing an average degree of polymerization;
b) a bis-secondary diamine residue, such as a piperazine derivative; c) a bis-primary diamine residue of formula —NH—Y—NH—, in which Y denotes a linear or branched hydrocarbon-based radical, or else the divalent radical —CH₂—CH₂—S—S—CH₂—CH₂—;
d) an ureylene group of formula —NH—CO—NH—.
   Preferably, X⁻ is an anion, such as chloride or bromide. These polymers have a number-average molar mass (Mn) generally of between 1000 and 100 000.

Mention may be made more particularly of polymers that are constituted of repeating units corresponding to the formula:

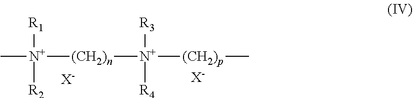

in which R₁, R₂, R₃ and R₄, which may be identical or different, denote an alkyl or hydroxyalkyl radical containing from 1 to 4 carbon atoms, n and p are integers ranging from 2 to 20, and X⁻ is an anion derived from a mineral or organic acid.

A particularly preferred compound of formula (IV) is the one for which R₁, R₂, R₃ and R₄ represent a methyl radical and n=3, p=6 and X═C₁, known as Hexadimethrine chloride according to the INCI (CTFA) nomenclature.

(9) polyquaternary ammonium polymers comprising units of formula (V):

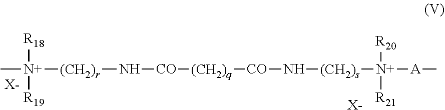

in which:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, in which p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X− denotes an anion such as a halide,
A denotes a divalent dihalide radical or preferably represents —CH₂—CH₂—O—CH₂—CH₂—.

Examples that may be mentioned include the products Mirapol® A 15, Mirapol® AD1, Mirapol® AZ1 and Mirapol®175 sold by the company Miranol.

(10) quaternary polymers of vinylpyrrolidone and of vinylimidazole, for instance the products sold under the names Luviquat® FC 905, FC 550 and FC 370 by the company BASF.
(11) polyamines such as Polyquart® H sold by Cognis, referred to under the name of Polyethylene glycol (15) tallow polyamine in the CTFA dictionary.
(12) polymers comprising in their structure:
(a) one or more units corresponding to formula (A) below:

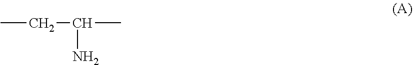

(b) optionally one or more units corresponding to formula (B) below:

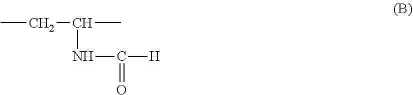

In other words, these polymers may especially be chosen from homopolymers or copolymers comprising one or more units derived from vinylamine and optionally one or more units derived from vinylformamide.

Preferably, these cationic polymers are chosen from polymers comprising, in their structure, from 5 mol % to 100 mol % of units corresponding to the formula (A) and from 0 to 95 mol % of units corresponding to the formula (B), preferentially from 10 mol % to 100 mol % of units corresponding to the formula (A) and from 0 to 90 mol % of units corresponding to the formula (B).

These polymers may be obtained, for example, by partial hydrolysis of polyvinylformamide. This hydrolysis may take place in acidic or basic medium.

The weight-average molecular weight of said polymer, measured by light scattering, may range from 1000 to 3 000 000 g/mol, preferably from 10 000 to 1 000 000 and more particularly from 100 000 to 500 000 g/mol.

The polymers comprising units of formula (A) and optionally units of formula (B) are sold in particular under the name Lupamin by the company BASF, for instance, in a non-limiting way, the products provided under the names Lupamin 9095, Lupamin 5095, Lupamin 1095, Lupamin 9030 (or Luviquat 9030) and Lupamin 9010.

Other cationic polymers that may be used in the context of the invention are cationic proteins or cationic protein hydrolyzates, polyalkyleneimines, in particular polyethyleneimines, polymers comprising vinylpyridine or vinylpyridinium units, condensates of polyamines and of epichlorohydrin, quaternary polyureylenes and chitin derivatives.

Preferably, the cationic polymers are chosen from those of families (1), (2), (7) and (10) mentioned above.

Among the cationic polymers mentioned above, the ones that may preferably be used are cationic polysaccharides, in particular cationic celluloses and cationic galactomannan gums, and in particular quaternary cellulose ether derivatives such as the products sold under the name JR 400 by the company Amerchol, cationic cyclopolymers, in particular dimethyldiallylammonium salt (for example chloride) homopolymers or copolymers, sold under the names Merquat 100, Merquat 550 and Merquat S by the company Nalco, quaternary polymers of vinylpyrrolidone and of vinylimidazole, optionally crosslinked homopolymers or copolymers of methacryloyloxy(C1-C4)alkyltri(C1-C4)alkylammonium salts, and mixtures thereof.

When they are present, the content of non-associative cationic polymer(s) in the composition according to the invention may range from 0.05% to 5% by weight relative to the total weight of the composition, preferably from 0.1% to 3% by weight and preferentially from 0.2% to 2% by weight relative to the total weight of the composition.

The cosmetic composition may also comprise one or more amphoteric polymers. For the purposes of the present invention, the term “amphoteric polymer” denotes any non-silicone (not comprising any silicon atoms) polymer containing cationic groups and/or groups that can be ionized into cationic groups and also anionic groups and/or groups that can be ionized into anionic groups.

The amphoteric polymers may preferably be chosen from amphoteric polymers comprising the repetition of:

(i) one or more units derived from a (meth)acrylamide-type monomer,
(ii) one or more units derived from a (meth)acrylamidoalkyltrialkylammonium-type monomer, and
(iii) one or more units derived from a (meth)acrylic acid-type acidic monomer.

Preferably, the units derived from a (meth)acrylamide-type monomer are units of structure (Ia) below:

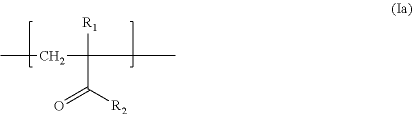

in which R₁ denotes H or CH₃ and R₂ is chosen from an amino, dimethylamino, tertbutylamino, dodecylamino and —NH—CH₂OH radical.

Preferably, said amphoteric polymer comprises the repetition of only one unit of formula (Ia).

The unit derived from a monomer of (meth)acrylamide type of formula (Ia) in which R₁ denotes H and R₂ is an amino radical (NH₂) is particularly preferred. It corresponds to the acrylamide monomer per se.

Preferably, the units derived from a monomer of (meth)acrylamidoalkyltrialkylammonium type are units of structure (IIa) below:

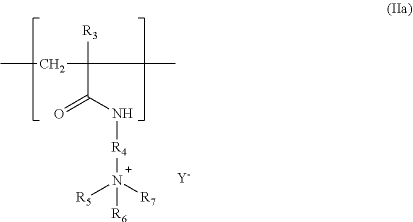

in which:
R₃ denotes H or CH₃,
R₄ denotes a group (CH₂)k, with k being an integer ranging from 1 to 6 and preferably from 2 to 4;
R₅, R₆ and R₇, which may be identical or different, denote a C₁-C₄ alkyl,
Y⁻ is an anion such as bromide, chloride, acetate, borate, citrate, tartrate, bisulfate, bisulfite, sulfate or phosphate.

Preferably, said amphoteric polymer comprises the repetition of only one unit of formula (IIa).

Among these units derived from a (meth)acrylamidoalkyltrialkylammonium-type monomer of formula (Ia), the ones that are preferred are those derived from the methacrylamidopropyltrimethylammonium chloride monomer, for which R₃ denotes a methyl radical, k is equal to 3, R₅, R₆ and R₇ denote a methyl radical, and Y⁻ denotes a chloride anion.

Preferably, the units derived from a monomer of (meth)acrylic acid type are units of formula (IIIa):

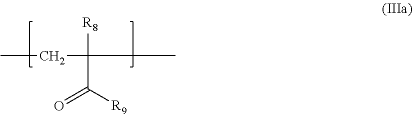

in which R₈ denotes H or CH₃ and R₉ denotes a hydroxyl radical or an —NH—C(CH₃)₂—CH₂—SO₃H radical.

The preferred units of formula (IIIa) correspond to the acrylic acid, methacrylic acid and 2-acrylamido-2-methylpropanesulfonic acid monomers.

Preferably, the unit derived from a monomer of (meth)acrylic acid type of formula (IIIa) is that derived from acrylic acid, for which R₈ denotes a hydrogen atom and R₉ denotes a hydroxyl radical.

The acidic monomer(s) of (meth)acrylic acid type may be non-neutralized or partially or totally neutralized with an organic or mineral base.

Preferably, said amphoteric polymer comprises the repetition of only one unit of formula (IIIa).

According to a preferred embodiment of the invention, the amphoteric polymer(s) of this type comprise at least 30 mol % of units derived from a monomer of (meth)acrylamide type (i). Preferably, they comprise from 30 mol % to 70 mol % and more preferably from 40 mol % to 60 mol % of units derived from a (meth)acrylamide-type monomer.

The content of units derived from a monomer of (meth)acrylamidoalkyltrialkylammonium type (ii) may advantageously be from 10 mol % to 60 mol % and preferentially from 20 mol % to 55 mol %.

The content of units derived from an acidic monomer of (meth)acrylic acid type (iii) may advantageously be from 1 mol % to 20 mol % and preferentially from 5 mol % to 15 mol %.

According to a particularly preferred embodiment of the invention, the amphoteric polymer of this type comprises:
from 30 mol % to 70 mol % and more preferably from 40 mol % to 60 mol % of units derived from a monomer of (meth)acrylamide type (i),
from 10 mol % to 60 mol % and preferentially from 20 mol % to 55 mol % of units derived from a monomer of (meth)acrylamidoalkyltrialkylammonium type (ii), and
from 1 mol % to 20 mol % and preferentially from 5 mol % to 15 mol % of units derived from a monomer of (meth)acrylic acid type (iii).

Amphoteric polymers of this type may also comprise additional units, other than the units derived from a (meth)acrylamide-type monomer, a (meth)acrylamidoalkyltrialkylammonium-type monomer and a (meth)acrylic acid-type monomer such as described above.

However, according to a preferred embodiment of the invention, said amphoteric polymers are constituted solely of units derived from monomers of (meth)acrylamide type (i), of (meth)acrylamidoalkyltrialkylammonium type (ii) and of (meth)acrylic acid type (iii).

Examples of particularly preferred amphoteric polymers that may be mentioned include acrylamide/methacrylamidopropyltrimethylammonium chloride/acrylic acid terpolymers. Such polymers are listed in the CTFA (INCI) dictionary under the name “Polyquaternium 53”. Corresponding products are in particular sold under the names Merquat 2003 and Merquat 2003 PR by the company Nalco.

As another type of amphoteric polymer that may be used, mention may also be made of copolymers based on (meth)acrylic acid and on a dialkyldiallylammonium salt, such as copolymers of (meth)acrylic acid and of dimethyldiallylammonium chloride. An example that may be mentioned is Merquat 280 sold by Nalco.

When they are present, the amphoteric polymer(s) may be present in the composition according to the invention in a total amount of between 0.05% and 5% by weight, preferably between 0.1% and 3% by weight, and more particularly between 0.2% and 2% by weight, relative to the total weight of the composition.

According to a preferred embodiment of the invention, the composition according to the invention comprises:
one or more anionic surfactants (i) chosen from polyoxyalkylenated alkyl(amido)ether carboxylic acids and salts thereof, preferably of formula (1) as defined above;
one or more anionic surfactants (ii) chosen from sodium, triethanolamine, magnesium or ammonium (C12-C14)alkyl sulfates and/or sodium, ammonium or magnesium (C12-C14)alkyl ether sulfates, which are oxyethylenated, for example with 1 to 10 mol of ethylene oxide;
one or more non-ionic surfactants of (C₆-C₂₄ alkyl)(poly)glycoside type, and more particularly (C₈-C₁₈ alkyl)(poly)glycosides;
one or more amphoteric surfactants chosen from (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines;
one or more associative cationic polymers chosen from the cationic polymers (B) derived from quaternized cellulose, particularly chosen from the hydroxyethylcelluloses of formula (Ib) and even better still polyquaternium-67.

The composition according to the invention may comprise water or a mixture of water and one or more cosmetically acceptable solvents chosen from C₁-C₄ alcohols, such as ethanol, isopropanol, tert-butanol or n-butanol; polyols such as glycerol, propylene glycol and polyethylene glycols; and mixtures thereof.

Preferably, the composition according to the invention has a total water content of between 50% and 95% by weight, preferably 60% and 90% by weight, even better still between 65% and 85% by weight relative to the total weight of the composition.

The pH of the compositions according to the invention generally ranges from 3.0 to 9.0, preferably from 3.5 to 7.0, preferentially from 4.0 to 6.0 and better still from 4.5 to 5.5.

The composition according to the invention may also comprise one or more standard additives that are well known in the art, such as natural or synthetic thickeners or viscosity regulators; C₁₂-C₃₀ fatty alcohols; ceramides; C₁₂-C₃₂ fatty esters such as isopropyl myristate, myristyl myristate, cetyl palmitate and stearyl stearate; mineral, plant or synthetic oils; vitamins or provitamins; non-ionic or anionic polymers; pH stabilizers, preservatives; dyes; fragrances; agents for preventing hair loss, antiseborrhoeic agents, antidandruff agents.

Those skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

Advantageously, the composition according to the invention does not comprise any silicone.

The compositions in accordance with the invention may be used as shampoos for washing and/or conditioning the hair; they are preferably applied in this case to damp hair in amounts that are effective for washing it; the lather generated by massaging or rubbing with the hands may then be removed, after an optional leave-on time, by rinsing with water, the operation possibly being repeated one or more times.

### Process and Use According to the Invention

Another subject of the present invention relates to a cosmetic treatment process, preferably a hair treatment process, in particular for washing and/or conditioning keratin materials, in particular human keratin fibres such as the hair, comprising the application to said materials of a composition as defined above, optionally followed by a leave-on time and/or rinsing and/or drying.

The composition may be applied to dry or wet hair, and preferably to wet or damp hair.

According to one embodiment, the process consists in applying to keratin fibres an effective amount of the composition according to the invention, optionally massaging the fibres, optionally leaving the composition to stand on said fibres, and rinsing. The leave-on time of the composition on the keratin fibres may be between a few seconds and 15 minutes and preferably between 30 seconds and 5 minutes. The composition is generally rinsed out with water.

An optional step of drying the keratin fibres may be performed.

The present invention also relates to the use of the composition according to the invention as described previously, for the cosmetic treatment of, in particular for washing and/or conditioning, keratin materials, in particular human keratin fibres such as the hair.

The examples that follow serve to illustrate the invention without, however, being limiting in nature.

### EXAMPLE 1

The cosmetic compositions according to the invention are prepared from the ingredients shown in the table below, the amounts of which are expressed as weight percentages of active material (AM).

| | Compositions (% AM) | | | |
|---|---|---|---|---|
| | A | B | C | D |
| Laureth-5 carboxylic acid | 2.0 | 2.0 | 2.0 | 3.0 |
| Sodium Laureth Sulfate | 7.0 | — | — | 7.0 |
| Sodium C₁₄-C₁₆ olefin sulfonate | — | 7.0 | 7.0 | — |
| Cocamidopropyl betaine | 5.0 | 6.4 | 6.4 | 7.0 |
| Caprylyl/capryl glucoside | — | 1.5 | 1.5 | 1.5 |
| Cocoyl glucoside | 1.1 | — | — | — |
| Polyquaternium-67 | 0.3 | 0.3 | 0.2 | 0.75 |
| NaCl | 1.0 | 1.1 | 1.2 | 1.3 |
| Preservatives | qs | qs | qs | qs |
| pH agent | qs pH | qs pH | qs pH | qs pH |
| | 4.7 ± 0.3 | 4.7 ± 0.3 | 5.1 ± 0.3 | 4.5 ± 0.3 |
| Water | qs 100 | qs 100 | qs 100 | qs 100 |
| Ratio* | 0.71 | 0.91 | 0.91 | 1 |

*Amphoteric surfactant (iv)/additional anionic surfactant (ii) ratio

| | Compositions (% AM) | | | | |
|---|---|---|---|---|---|
| | E | F | G | H | I |
| Laureth-5 carboxylic | 2.0 | 3.0 | 3.0 | 6.0 | 3.0 |
| acid | | | | | |
| Sodium Laureth Sulfate | — | 7.0 | 7.0 | 7.0 | 7.0 |
| Sodium C₁₄-C₁₆ olefin | 7.0 | — | — | — | — |
| sulfonate | | | | | |
| Cocamidopropyl betaine | 6.4 | 7.0 | 7.0 | 7.0 | 7.0 |
| Caprylyl/capryl glucoside | 1.5 | 1.5 | 1.5 | 3.0 | 1.5 |
| Polyquaternium-67 | 0.2 | 0.3 | 0.3 | 0.5 | 0.75 |
| Polyquaternium-53 | — | — | — | 0.3 | — |
| Propylene glycol | — | 0.24 | — | — | — |
| PEG-55 propylene | — | 0.24 | — | — | — |
| glycol oleate | | | | | |
| PPG-5-Ceteth-20 | — | 0.7 | — | — | — |
| Plant oil (coconut | 0.03 | — | — | — | — |
| in particular) | | | | | |
| Glycerol | — | — | 1.5 | — | — |
| Glycol distearate | — | — | 0.6 | — | — |
| NaCl | 1.2 | 1.3 | 1.3 | 1.8 | 1.3 |
| Preservatives | qs | qs | qs | qs | qs |
| pH agent | qs pH | qs pH | qs pH | qs pH | qs pH |
| | 5.1 ± 0.3 | 4.7 ± 0.3 | 4.5 ± 0.3 | 4.5 ± 0.3 | 4.5 ± 0.2 |
| Water | qs 100 | qs 100 | qs 100 | qs 100 | qs 100 |
| Ratio* | 0.91 | 1 | 1 | 1 | 1 |

*Amphoteric surfactant (iv)/additional anionic surfactant (ii) ratio

The compositions according to the invention may be used as shampoos.

It is noted that they make it possible to provide good disentangling, smooth feel and visual smoothness, suppleness and fibre-coating properties, better than those provided by products on the market (DOP shampoo for example).

### EXAMPLE 2

The composition according to the invention I prepared above is compared with a comparative composition not comprising any associative polymer.

| | Compositions (% AM) | |
|---|---|---|
| | Invention | Comparative |
| Laureth-5 carboxylic acid | 3.0 | 6.0 |
| Sodium Laureth Sulfate | 7.0 | 7.0 |
| Cocamidopropyl betaine | 7.0 | 7.0 |
| Caprylyl/capryl glucoside | 1.5 | — |
| Polyquaternium-67 | 0.75 | — |
| Polyquaternium-53 | — | 0.3 |
| Polyquaternium-6 | — | 0.5 |
| Laureth-4 | — | 1.0 |
| Laureth-12 | — | 4.0 |
| NaCl | 1.3 | 1.8 |
| Preservatives | qs | qs |
| Water | qs 100 | qs 100 |

The compositions were applied per ½ head to the heads of 6 volunteers with sensitized hair, in a proportion of 6 g of composition per ½ head, they were then rinsed off and the hair was dried.

A sensory evaluation was carried out, blind, by an expert who assigned a score ranging from 0 (very bad) to 5 (very good) for the criterion tested, in increments of 0.5.

The following results are obtained:

On damp hair

| | | | Smooth | |
|---|---|---|---|---|
| | Disentangling | Coating | feel | Suppleness |
| Invention | 3.9 | 3.9 | 3.9 | 3.8 |
| Comparative | 3.2 | 3.1 | 2.9 | 3.1 |
| p-value | 0.025 | 0.005 | 0.004 | 0.012 |

On dry hair

| | | Visual | | Smooth |
|---|---|---|---|---|
| | Disentangling | smoothness | Coating | feel |
| Invention | 3.6 | 3.0 | 3.6 | 3.5 |
| Comparative | 3.2 | 2.4 | 2.6 | 2.6 |
| p-value | 0.078 | 0.030 | 0.004 | 0.000 |

It is noted that the composition according to the invention provides better performance qualities in terms of conditioning than the comparative composition.

### EXAMPLE 3

A composition according to the invention and a comparative composition below are prepared.

| | Compositions (% AM) | |
|---|---|---|
| | Invention | Comparative |
| Laureth-5 carboxylic acid | 2 | 2 |
| Sodium Laureth Sulfate | 7 | 7 |
| Cocamidopropyl betaine | 5 | 2.8 |
| Cocoyl glucoside | 1.15 | 1.15 |
| Polyquaternium-67 | 0.30 | 0.30 |
| Preservatives | qs | qs |
| Water | qs 100 | qs 100 |
| (iv)/(ii) Ratio | 0.7 | 0.4 |

The compositions were applied to locks of sensitized hair (SA20), in a proportion of 1 g of composition per lock of 2.7 g, then the locks were rinsed.

A sensory evaluation was carried out, blind, by 7 experts who assigned, for the criterion tested, a score ranging from 0 (very bad) to 5 (very good) in increments of 0.5.

To evaluate the disentangling, the expert slides a comb through the hair from the root to the end, and evaluates the ease with which it passes through.

To evaluate the smoothness, the expert takes the lock of hair between their fingers and slides the fingers over the hair from the root to the end. The expert evaluates whether the hair has bumps and catches on the fingers.

The following results are obtained:

| | Disentangling | | Smooth Feel | |
|---|---|---|---|---|
| | Invention | Comparative | Invention | Comparative |
| Expert 1 | 5 | 3.5 | 5 | 3.5 |
| Expert 2 | 5 | 4 | 5 | 3 |
| Expert 3 | 5 | 4 | 5 | 4 |
| Expert 4 | 4.5 | 4.5 | 4.5 | 3.5 |
| Expert 5 | 5 | 4.5 | 4 | 3.5 |
| Expert 6 | 5 | 4 | 4 | 4.5 |
| Expert 7 | 4 | 3 | 4 | 3 |
| Mean | 4.8 | 3.9 | 4.5 | 3.6 |

| | | |
|---|---|---|
| p-value | 0.007 | 0.011 |

The composition according to the invention provides better performance qualities in terms of disentangling and smooth feel than the comparative composition.

According to the Mann-Whitney test for independent samples, the calculation of the p-value gives values for the p-value of <0.05.

The composition according to the invention is thus significantly different, in terms of disentangling and smooth feel, from the comparative composition.

1.-19. (canceled)

20. A cosmetic composition comprising:
(i) at least one anionic surfactant chosen from polyoxyalkylenated alkyl(amido)ether carboxylic acids or salts thereof;
(ii) at least one anionic surfactant other than the at least one anionic surfactant chosen from polyoxyalkylenated alkyl(amido)ether carboxylic acids and salts thereof (i);
(iii) at least one non-ionic surfactant;
(iv) at least one amphoteric surfactant; and
(v) at least one cationic associative polymer,
wherein the weight ratio of the at least one amphoteric surfactant (iv) to the at least one anionic surfactant (ii) other than the polyoxyalkylenated alkyl(amido)ether carboxylic acids and salts thereof is greater than or equal to 0.5.

21. The composition of claim 20, wherein the polyoxyalkylenated alkyl(amido)ether carboxylic acids are chosen from compounds of formula (1):
R₁OC₂H₄)ₙ—OCH₂COOA (1)
wherein in formula (1):
R1 is chosen from a linear or branched C₆-C₂₄ alkyl or alkenyl radical, a (C₈-C₉)alkylphenyl radical, or a radical R₂CONH—CH₂—CH₂— with R2 denoting a linear or branched C₉-C₂₁ alkyl or alkenyl radical;
n is an integer or decimal number ranging from 2 to 24; and
A is chosen from H, ammonium, Na, K, Li, Mg, Ca, a monoethanolamine residue, or triethanolamine residue.

22. The composition of claim 20, wherein the polyoxyalkylenated alkyl(amido)ether carboxylic acids are chosen from compounds of formula (1):
R₁OC₂H₄)ₙ—OCH₂COOA (1)
wherein in formula (1):
R1 is chosen from a C12-C14 alkyl, cocoyl, oleyl, nonylphenyl, or octylphenyl radical,
A denotes a hydrogen or sodium atom, and
n ranges from 2 to 20.

23. The composition of claim 20, wherein the at least one anionic surfactant chosen from polyoxyalkylenated alkyl(amido)ether carboxylic acids or salts thereof is present in a total amount ranging from 0.1% to 30% by weight, relative to the total weight of the composition.

24. The composition of claim 20, wherein the at least one anionic surfactant (ii) is chosen, alone or as a mixture, from:
C₆-C₂₄ and especially C₁₂-C₂₀ alkyl sulfates;
C₆-C₂₄ alkyl ether sulfates;
C₆-C₂₄ olefin sulfonates;
C₆-C₂₄ alkylsulfosuccinates;
C₆-C₂₄ alkyl ether sulfosuccinates;
(C₆-C₂₄) acylisethionates;
C₆-C₂₄ acylsarcosinates;
C₆-C₂₄ acylglutamates;
C₆-C₂₄ acylglycinates;
optionally in the form of alkali metal, alkaline-earth metal, ammonium salts, or amino alcohol salts.

25. The composition of claim 20, wherein the at least one anionic surfactant (ii) is present in a total amount ranging from 3% to 20% by weight, relative to the total weight of the composition.

26. The composition of claim 20, wherein the at least one non-ionic surfactant is chosen from:
alcohols, α-diols, and (C₁₋₂₀)alkylphenols, wherein the alcohols, α-diols, and (C₁-20)alkylphenols being polyethoxylated, polypropoxylated, and/or polyglycerolated, the number of ethylene oxide and/or propylene oxide groups optionally ranging from 1 to 100, and the number of glycerol groups optionally ranging from 2 to 30; alternatively, the alcohols, α-diols, and (C₁₋₂₀)alkylphenols comprising at least one fatty chain comprising from 8 to 40 carbon atoms;
condensates of ethylene oxide and propylene oxide with fatty alcohols;
polyethoxylated fatty amides containing from 2 to 30 ethylene oxide units, and polyglycerolated fatty amides including on average from 1 to 5 glycerol groups;
ethoxylated fatty acid esters of sorbitan;
fatty acid esters of sucrose;
polyoxyalkylenated, preferably polyoxyethylenated, or fatty acid esters containing from 2 to 150 mol of ethylene oxide, including oxyethylenated plant oils;
N—(C6-24 alkyl)glucamine derivatives;
amine oxides or N—(C10-14 acyl)aminopropylmorpholine oxides;
non-ionic surfactants of alkyl(poly)glycoside type, represented by the following general formula: R1O—(R2O)t-(G)v,
wherein:
R1 is chosen from a linear or branched alkyl or alkenyl radical including 6 to 24 carbon atoms, or an alkylphenyl radical of which the linear or branched alkyl radical comprises 6 to 24 carbon atoms;
R2 represents an alkylene radical including 2 to 4 carbon atoms;
G represents a sugar unit including 5 to 6 carbon atoms;
t denotes a value ranging from 0 to 10; and
v denotes a value ranging from 1 to 15.

27. The composition of claim 20, wherein the at least one non-ionic surfactant is chosen from, alone or as a mixture, (C₆-C₂₄ alkyl)(poly)glycosides.

28. The composition of claim 20, wherein the at least one non-ionic surfactant is present in a total amount ranging from 0.1% to 15% by weight, relative to the total weight of the composition.

29. The composition of claim 20, wherein the at least one amphoteric surfactant is chosen from:
(C₈-C₂)alkylbetaines, (C₈-C₂₀)alkylsulfobetaines, (C₈-C₂₀)alkylamido(C₃-C₈)alkylbetaines, or (C₈-C₂₀)alkylamido(C₆-C₈)alkylsulfobetaines;
compounds having respective formulae (II) and (III) below:
Rₐ—CONHCH₂CH₂—N⁺(R_{b})(R_{c})—CH₂COO⁻,M⁺,X⁻ (II)
wherein in formula (II):
Rₐ is chosen form a C₁₀ to C₃₀ alkyl or alkenyl group derived from an acid RₐCOOH, a heptyl group, a nonyl group, or an undecyl group;
R_{b} is chosen from a β-hydroxyethyl group; and
R_{c} is chosen from a carboxymethyl group;
M⁺ represents a cationic counterion derived from an alkali metal, an alkaline-earth metal, an ammonium ion, or an ion derived from an organic amine; and
X⁻ represents an organic or mineral anionic counterion; or
alternatively M⁺ and X⁻ are absent;
R_{a′}—CONHCH₂CH₂—N(B)(B′) (III)
wherein in formula (III):
B is chosen from the group —CH₂CH₂OX′;
B′ is chosen from the group —(CH₂)_{z}Y′, wherein z=1 or 2;
X′ is chosen from the group —CH₂COOH, —CH₂—COOZ′, —CH₂CH₂COOH, CH₂CH₂—COOZ′, or a hydrogen atom;
Y′ is chosen from the group —OOH, —COOZ′, —CH₂CH(OH)SO₃H, or CH₂CH(OH)SO₃—Z′;
Z′ represents a cationic counterion derived from an alkali metal, an alkaline-earth metal, an ammonium ion, or an ion derived from an organic amine;
R_{a′} is chosen from a C₁₀ to C₃₀ alkyl or alkenyl group of an acid R_{a′}—COOH, an alkyl group or its iso form, or an unsaturated C₁₇ group;
compounds of formula (IV):
R_{a″}—NHCH(Y″)—(CH₂)ₙCONH(CH₂)_{n′}—N(R_{d})(Rₑ) (IV)
wherein in formula (IV):
Y″ is chosen from the group —OOH, —COOZ″, —CH₂—CH(OH)SO₃H, or CH₂CH(OH)SO₃—Z″;
R_{d} and Rₑ represent, independently of one another, a C₁ to C₄ alkyl or hydroxyalkyl radical;
Z″ represents a cationic counterion derived from an alkali metal or alkaline-earth metal, an ammonium ion, or an ion derived from an organic amine;
R_{a″} represents a C₁₀ to C₃₀ alkyl or alkenyl group of an acid R_{a″}-COOH;
n and n′ denote, independently of one another, an integer ranging from 1 to 3.

30. The composition of claim 20, wherein the at least one amphoteric surfactant is present in a total amount ranging from 3% to 20% by weight, relative to the total weight of the composition.

31. The composition of claim 20, wherein the weight ratio of the total amount of the at least one amphoteric surfactant (iv) to the total amount of the at least one anionic surfactant (ii) other than the at least one anionic surfactant chosen from polyoxyalkylenated alkyl(amido)ether carboxylic acids or salts thereof (i) is greater than or equal to 0.5.

32. The composition of claim 20, wherein the at least one cationic associative polymer is chosen, alone or as a mixture, from:
(A) cationic associative polyurethanes, which can be represented by general formula (Ia) below:
R—X—(P)n-[L-(Y)m]r-L′-(P′)p—X′—R′
wherein:
R and R′, which may be identical or different, represent a hydrophobic group or a hydrogen atom;
X and X′, which may be identical or different, is chosen from a group comprising an amine function optionally bearing a hydrophobic group, or alternatively the group L″;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100 inclusive;
n, m and p are each, independently of one another, between 0 and 1000;
the cationic associative polyurethanes containing at least one protonated or quaternized amine function and at least one hydrophobic group.
(B) quaternized cellulose derivatives;
(C) cationic polyvinyllactams comprising:
a) at least one monomer of vinyllactam or alkylvinyllactam type; or
b) at least one monomer of formula (Ic) or formula (IIc) below: 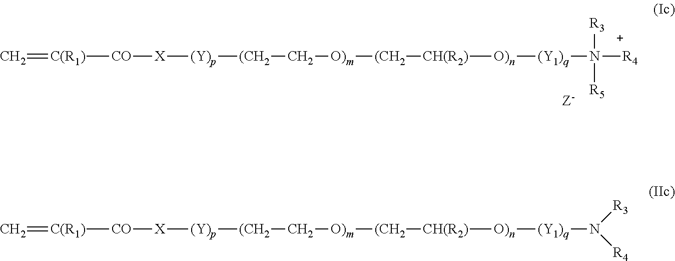
wherein in formula (Ic) and formula (IIc):
X is chosen from an oxygen atom or an NR₆ radical,
R₁ and R₆ are chosen from, independently of one another, a hydrogen atom or a linear or branched C₁-C₅ alkyl radical,
R₂ is chosen from a linear or branched C₁-C₄ alkyl radical,
R₃, R₄, and R₅ are chosen from, independently of one another, a hydrogen atom, a linear or branched C₁-C₃₀ alkyl radical, or a radical of formula (IIIc):
—(Y₂)ᵣ—(CH₂—CH(R₇)—O)ₓ—R₈ (IIIC)
Y, Y₁ and Y₂ are chosen from, independently of one another, a linear or branched C₂-C₁₆ alkylene radical,
R₇ is chosen from a hydrogen atom, a linear or branched C₁-C₄ alkyl radical, or a linear or branched C₁-C₄ hydroxyalkyl radical,
R₈ is chosen from a hydrogen atom, a linear or branched C₁-C₃₀ alkyl radical,
p, q and r denote, independently of one another, either the value zero or the value 1,
m and n denote, independently of one another, an integer ranging from 0 to 100,
x denotes an integer ranging from 1 to 100 inclusive,
Z denotes an anionic counterion of an organic or mineral acid,
with the proviso that:
at least one of the substituents R₃, R₄, R₅ or R₈ denotes a linear or branched C₉-C₃₀ alkyl radical,
if m and/or n is other than zero, then q is equal to 1,
if m=n=0, then p or q equals 0.
(D) cationic polymers obtained by polymerization of a monomer mixture comprising at least one vinyl monomer substituted with at least one amino group, at least one hydrophobic non-ionic vinyl monomer, and/or at least one associative vinyl monomer.

33. The composition of claim 32, wherein the quaternized celluloses are modified with groups comprising at least one fatty chain, wherein the at least one fatty chain incudes at least 8 carbon atoms.

34. The composition of claim 20, wherein the at least one cationic associative polymer is chosen from the quaternized hydroxyethylcelluloses of formula (Ib): 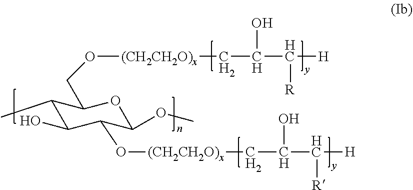
wherein in formula (Ib):
R represents an ammonium group RaRbRcN⁺—, Q⁻, wherein Ra, Rb, and Rc, which may be identical or different, are chosen from a hydrogen atom, or a linear or branched C₁-C₃₀ alkyl, and Q⁻ represents an anionic counterion;
R′ represents an ammonium group R′aR′bR′cN⁺—, Q′, wherein R′a, R′b, and R′c, which may be identical or different, are chosen from a hydrogen atom, or a linear or branched C₁-C₃₀ alkyl, and Q′⁻ represents an anionic counterion;
wherein at least one of the Ra, Rb, Rc, Ra, R′b, and R′c radicals represents a linear or branched C₈-C₃₀alkyl;
n, x and y, which may be identical or different, represent an integer between 1 and 10 000.

35. The composition of claim 34, wherein only one of the Ra, Rb, Rc, R′a, R′b, and R′c radicals represents a linear or branched C₈-C₃₀ alkyl.

36. The composition of claim 20, wherein the at least one cationic associative polymer is present in a total amount ranging from 0.01% to 8% by weight, relative to the total weight of the composition.

37. The composition of claim 20, further comprising at least one cationic polymer, wherein the cationic charge density of the at least one cationic polymer is greater than or equal to 3 milliequivalents/gram (meq/g); and wherein the at least one cationic polymer is present in a total amount ranging from 0.05% to 5% by weight, relative to the total weight of the composition.

38. The composition of claim 20, further comprising at least one amphoteric polymer, wherein the at least one amphoteric polymer comprises the repetition of:
(i) at least one unit derived from a (meth)acrylamide-type monomer,
(ii) at least one unit derived from a (meth)acrylamidoalkyltrialkylammonium-type monomer, and
(iii) at least one unit derived from a (meth)acrylic acid-type acid monomer;
wherein the at least one amphoteric polymer is present in a total amount ranging from 0.05% to 5% by weight, relative to the total weight of the composition.

39. A method of treating keratin materials, comprising applying to the keratin materials an effective amount of a composition comprising:
(i) at least one anionic surfactant chosen from polyoxyalkylenated alkyl(amido)ether carboxylic acids or salts thereof;
(ii) at least one anionic surfactant other than the at least one anionic surfactant chosen from polyoxyalkylenated alkyl(amido)ether carboxylic acids and salts thereof (i);
(iii) at least one non-ionic surfactant;
(iv) at least one amphoteric surfactant; and
(v) at least one cationic associative polymer,
wherein the weight ratio of the at least one amphoteric surfactant (iv) to the at least one anionic surfactant (ii) other than the polyoxyalkylenated alkyl(amido)ether carboxylic acids and salts thereof is greater than or equal to 0.5.
optionally, subsequently rinsing the keratin materials after the composition is left on the keratin materials for a desired period of time; and
optionally, drying the keratin materials after rinsing the keratin materials.
